(19) United States
(12) Patent Application Publication
(10) Pub. No.: US 2022/0152338 A1
(43) Pub. Date: May 19, 2022

(54) AIRWAY THERAPY SYSTEM

(71) Applicant: KONINKLIJKE PHILIPS N.V., EINDHOVEN (NL)

(72) Inventors: Pascal de Graaf, Eindhoven (NL); Rita Priori, Utrecth (NL); Samer Bou Jawde, Boston, MA (US); Kiran Hamilton J. Dellimore, Utrecth (NL); Marco Baragona, Delft (NL)

(21) Appl. No.: 17/463,695

(22) Filed: Sep. 1, 2021

Related U.S. Application Data
(60) Provisional application No. 63/114,807, filed on Nov. 17, 2020.

(51) Int. Cl. A61M 16/20; A61M 16/00

(52) U.S. Cl. CPC A61M 16/205; A61M 16/00; A61M 16/204; A61M 2230/62; A61M 2016/0027; A61M 2230/46; A61M 2230/40; A61M 2205/3303

(57) ABSTRACT

The invention provides a positive airway pressure therapy system comprising a positive pressure generation unit, adapted to generate a positive pressure airflow for provision to a subject, and an oscillatory pressure generation unit adapted to modulate the positive pressure airflow at a modulation frequency thereby imparting a frequency component to the positive pressure air flow. The oscillatory pressure generation unit is adapted to modulate the airflow during an exhalation phase of a breathing cycle of the subject.

### CROSS-REFERENCE TO RELATED APPLICATIONS

This patent application claims the priority benefit under 35 U.S.C. § 119(e) of U.S. Provisional Application No. 63/114,807, filed on Nov. 17, 2020, the contents of which are herein incorporated by reference.

### FIELD OF THE INVENTION

The invention relates the field of airway therapy systems, and more specifically to the field of positive airway pressure therapy systems.

### BACKGROUND OF THE INVENTION

People affected by respiratory diseases such as chronic obstructive pulmonary disease (COPD) or bronchiectasis often show excessive mucus production. Indeed, night-time disturbances and morning symptoms of COPD are highly prevalent and often associated with increased mucus production. Airway clearance devices may be used to mobilize and remove mucus, when coughing alone is not sufficient; however, they cannot be used at night or when a subject is undergoing other therapies such as non-invasive ventilation (NIV).

In conditions where the lung's normal airway clearance mechanism is not sufficiently functional (e.g. in bronchiectasis), mucus can accumulate leading to infections and poor perfusion. One method to remove this mucus from the airways is Oscillating Positive Expiratory Pressure (OPEP) therapy. To be effective, this therapy requires up to four 20-minute sessions each day with a hand-held device, which is time consuming, places a considerable burden on the subject and increases the likelihood that the subject will not be fully adherent to the OPEP therapy.

Another method of airway clearance is intrapulmonary percussive ventilation (IPV), which is a pressure-limited, time-cycled, high-frequency mode of ventilation that delivers sub-physiologic tidal volumes. IPV is typically used in hospital as a dedicated airway clearance technique delivered through a face mask, mouthpiece, endotracheal tube, or tracheostomy.

In a cross-sectional study about symptom variability in subjects with COPD, phlegm was the second most common symptom after breathlessness. Further, more than half of the subjects in the study reported phlegm to be mostly problematic on waking, compared to later in the day where it is almost negligible in comparison. This suggests that mucus tends to accumulate over the night and that an intervention is needed, in order to alleviate symptoms and decrease night time disturbances.

There is therefore a need for a mucus loosening solution with minimal subject burden, which supports subjects in easily maintaining their therapy adherence.

### SUMMARY OF THE INVENTION

The invention is defined by the claims.

According to examples in accordance with an aspect of the invention, there is provided a positive airway pressure therapy system comprising:

a positive pressure generation unit adapted to generate a positive pressure airflow for provision to a subject; and

an oscillatory pressure generation unit adapted to modulate the positive pressure airflow at a modulation frequency thereby imparting a frequency component to the positive pressure air flow to be provided to the subject, wherein the oscillatory pressure generation unit is adapted to modulate the airflow during an exhalation phase of a breathing cycle of the subject.

Proposed is a concept of combining positive airway pressure therapy with oscillatory pressure generation. In this way, intra-airway pressure oscillations may be provided together with positive airway pressure, which may help to thin mucus and decrease phlegm-associated symptoms, for example.

Embodiments may therefore be based on a combination of OPEP therapy (or IPV therapy) with positive airway pressure therapy (such as Continuous Positive Airway Pressure (CPAP) therapy or Non-Invasive Ventilation (NIV) therapy)). Through such combination, benefits associated with OPEP/IPV therapy may be enhanced. For instance, the CPAP therapy may avoid collapse of parts of the subject's airways, thus improving effectiveness of concurrent OPEP/IPV therapy.

Purely by way of illustration, proposed embodiments may be thought of as superimposing (pressure) oscillations onto a CPAP airflow. For instance, a positive pressure airflow for provision to a subject may have (relatively) small amplitude oscillations applied, thus generating a continuous but oscillating positive airway pressure airflow.

A proposed system provides a means of simultaneously generating oscillations in an airway of the subject and providing positive airway pressure therapy, thereby loosening the mucus in the airway of the subject during positive airway pressure therapy. In this way, oscillatory pressure therapy for loosening the mucus in the airway of the subject does not need to be performed separately by the subject and may be performed as part of an existing positive airway pressure therapy regime. Thus, the burden of the subject to perform oscillatory pressure therapy is reduced.

Further, as positive airway pressure therapy is typically administered at night, the provision of simultaneous oscillatory pressure therapy aids in loosening the mucus in the airway of the subject when mucus based symptoms are typically at their worst. Accordingly, the effectiveness of the oscillatory pressure therapy may also be increased.

In an embodiment, the oscillatory pressure generation unit may be further adapted to modulate the airflow during a predetermined sleep stage of the subject. In this way, the disturbances to the subject during sleep may be reduced by controlling the modulation of the airflow based on the sleep stage of the subject.

In a further embodiment, the oscillatory pressure generation unit may be further adapted to generate a sleep trend over a plurality of sleep sessions, and the oscillatory pressure generation unit may be further adapted to adjust modulation of the airflow based on the sleep trend. Historical data relating to the user's sleep pattern may thus be used as a basis for modulating the airflow. Further, the generated sleep trend may be used to measure the progress of the disease over time.

In an embodiment, the modulation frequency may comprise a range of frequencies. This may facilitate the use of an optimal modulation frequency for a given subject/user. For instance, an optimal modulation frequency for a specific subject may be determined and then used in modulating the airflow.

In some embodiments, the oscillatory pressure generation unit may comprise an actuator valve, and the actuator valve may be adapted to actuate at the modulation frequency thereby imparting the frequency component to the airflow. In this way, the oscillatory pressure generation unit may modulate the airflow without requiring any change in the behavior of the positive pressure generation unit. For instance, a simple and cheap mechanism may be added to modulate the airflow output from the positive pressure generation unit, thus avoiding the need for any modification to the positive pressure generation unit.

In an embodiment, the oscillatory pressure generation unit may further comprise a pressure sensor in communication with the airflow, wherein the pressure sensor is adapted to obtain a pressure measurement from the airflow, and wherein the oscillatory pressure generation unit is adapted to adjust the modulation of the airflow based on the pressure measurement. This may allow for precise control of the modulation of the airflow (e.g. according to the output of the pressure sensor).

In an embodiment, the oscillatory pressure generation unit may be adapted to control the positive pressure generation unit to modulate the airflow. In this way, the oscillatory pressure generation unit may modulate the airflow by controlling the operation of the positive pressure generation unit.

In an embodiment, the oscillatory pressure generation unit and the positive pressure generation unit are integrated into a ventilation device.

In an embodiment, the positive pressure generation unit may be further adapted to increase the generated airflow when the subject is in an inhalation phase of the breath cycle.

In this way, the exhalation phase of the subject is increased, which results in more time for the delivery of the oscillatory pressure therapy.

In an embodiment, the subject ventilation device may comprise a subject measurement unit adapted to acquire a measure from the subject. For example, the subject measurement unit is adapted to obtain an efficacy measure of the modulated airflow from the subject, and wherein the oscillatory pressure generation unit is adapted to adjust the modulation of the airflow based on the efficacy measure. The modulation of the airflow may therefore be controlled based on the effectiveness of the oscillatory pressure therapy delivered to the subject. Variable and/or dynamic oscillatory pressure generation may therefore be achieved by proposed embodiments.

In an embodiment, the subject measurement unit may be adapted to obtain a measure of airway resistance over time from an airway of the subject, and the oscillatory pressure generation unit may then be adapted to modulate the airflow based on the measure of airway resistance. For instance, a build-up of mucus may be detected based on the airway resistance over time and used to control the modulation of the airflow. Dynamic control concepts may therefore be employed by embodiments so as to achieve improved functionality and/performance.

In some embodiments, the subject measurement unit may be adapted to obtain a respiratory mechanics measure from an airway of the subject, and the oscillatory pressure generation unit may be adapted to adjust the modulation of the airflow based on the respiratory mechanics measure. In this way, the behavior of the respiratory system may be taken into account and used to more accurately control the modulation of the airflow.

The subject measurement unit may be adapted to obtain a measurement of a posture of the subject, and the oscillatory pressure generation unit may be adapted to adjust the modulation of the airflow based on the posture of the subject. In this way, the posture of the subject at which the oscillatory pressure therapy is most effective may be identified.

The positive airway pressure therapy system may comprise one or more of: a CPAP device; a BiPAP device; and an NIV device. A conventional positive airway pressure therapy system may therefore be employed by proposed embodiments. Further, proposed embodiments may be configured such that a conventional positive airway pressure therapy system is adapted to provide/deliver OPEP or IPV therapy, thus alleviating a requirement for a subject to use two separate devices/systems. Embodiments may thus help to provide improved or extended functionality in positive airway pressure therapy systems.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a simplified block diagram of a positive airway pressure therapy system according to an exemplary embodiment;

FIG. 2A is a simplified block diagram of a positive airway pressure therapy system according to an embodiment, wherein an oscillatory pressure generation unit is integrated into a device along with a positive pressure generation unit;

FIG. 2B is a simplified block diagram of a positive airway pressure therapy system according to an alternative embodiment, wherein the oscillatory pressure generation unit is provided as a separate device with respect to the positive pressure generation unit;

FIG. 3 depicts a modification to the embodiment of FIG. 2A; and

FIG. 4 is a flow diagram of a positive airway pressure therapy method according to an embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

It should be understood that the Figures are merely schematic and are not drawn to scale. It should also be understood that the same reference numerals are used throughout the Figures to indicate the same or similar parts.

Implementations in accordance with the present disclosure relate to various techniques, methods, schemes and/or solutions pertaining to supplementing or improving positive airway pressure therapy. According to proposed concepts, a number of possible solutions may be implemented separately or jointly. That is, although these possible solutions may be described below separately, two or more of these possible solutions may be implemented in one combination or another.

In particular, proposed concepts may be based on the realisation that oscillatory pressure generation may be combined with positive airway pressure therapy. Such combination may, for example, help to loosen and thin mucus and decrease phlegm-associated symptoms for a subject.

By way of example, embodiments may therefore be based on a combination of OPEP therapy (or IPV therapy) with positive airway pressure therapy (such as Continuous Positive Airway Pressure (CPAP) therapy, Bi-level Positive Airway Pressure (BiPAP) or Non-Invasive Ventilation (MV) therapy)) to enhance benefits associated with OPEP/IPV therapy. Investigations have shown that CPAP therapy may avoid collapse of parts of the subject's airways, thereby improving effectiveness of concurrent OPEP/IPV therapy.

Proposed embodiments may therefore provide an approach to combining OPEP/IPV therapy with CPAP/NIV/BiPAP therapy. Accordingly, embodiments may provide improved respiratory support and/or therapy.

By way of example, according to one embodiment, there is provided a positive airway pressure therapy system comprising a positive pressure generation unit that is configured to generate a positive pressure airflow for provision to a subject. The system further comprises an oscillatory pressure generation unit that is configured to modulate the positive pressure airflow at a modulation frequency thereby imparting a frequency component to the positive pressure air flow. In particular, the oscillatory pressure generation unit is adapted to modulate the airflow during an exhalation phase of a breathing cycle of the subject. The system thus superimposes an oscillatory vibration on a positive pressure airflow that, during an exhalation of a subject, may help to loosen and clear mucus in the airways of the subject.

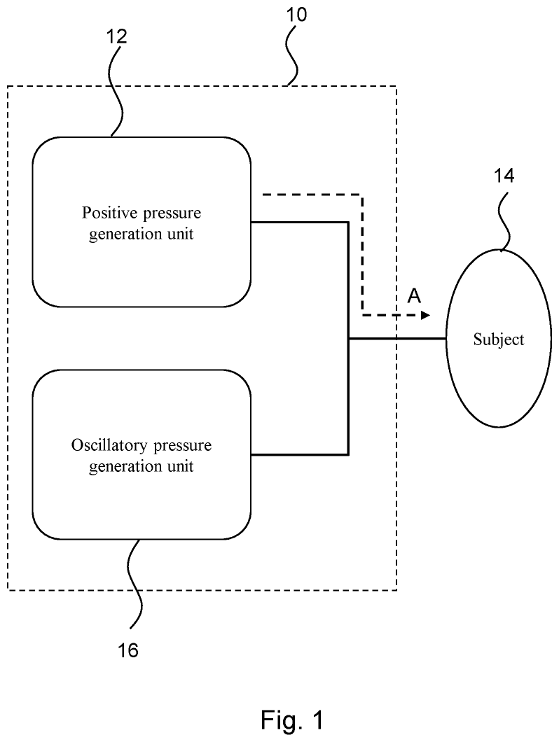

Referring to FIG. 1, there is depicted a simplified block diagram of a positive airway pressure therapy system 10 according to an exemplary embodiment. The system 10 comprises a positive pressure generation unit 12 that is adapted to generate a positive pressure airflow (indicated by the dashed arrow labelled ‘A’) for provision to a subject 14. The system 10 also comprises an oscillatory pressure generation unit 16 that is adapted to modulate the positive pressure airflow at a modulation frequency, thereby imparting a frequency component to the positive pressure air flow to be provided to the subject.

Specifically, the oscillatory pressure generation unit 16 is adapted to modulate the airflow during an exhalation phase of a breathing cycle of the subject.

Furthermore, the oscillatory pressure generation unit 16 is further adapted to modulate the airflow during a predetermined sleep stage of the subject. The oscillatory pressure generation unit 16 is also adapted to generate a sleep trend over a plurality of sleep sessions of the subject, and the oscillatory pressure generation unit 16 is configured to adjust modulation of the airflow based on the sleep trend.

The embodiment of FIG. 1 is enhanced with the inclusion of sleep staging. In this way, the positive airway pressure therapy can be provided during sleep (e.g. at times where the user's arousal threshold is sufficiently high (e.g. deep sleep)) and/or it can be stopped if it is causing less deep sleep or even awakenings.

Sleep stage information can be obtained in a number of ways, e.g. by measuring with electrodes on the body, with accelerometers or PPG sensors in a wearable device or by analysing the respiration pattern through the airflow signal from the Positive Airway Pressure (PAP) device itself. Indeed, various different approaches to obtaining sleep stage information are known and widely used. Detailed description of obtaining sleep stage information is therefore omitted from this description.

By performing therapy during sleep, a subject (i.e. user of the system) can benefit from airway clearance therapy over a relatively long period of time (e.g. an entire sleep period that may extend across multiple hours), during which conventional therapy and medication cannot typically be used. In this way, the subject may be relieved from performing the IPV/OPEP therapy during waking hours, thus improving overall quality of life and therapy adherence. Additionally, it may facilitate therapy performance with parameters that would not normally be accepted by the subject, thus enhancing therapy effectiveness. For example, the therapy time may be lengthened/extended. Also, higher positive pressures may be employed during the inhalation phase to increase the expiratory volume. That is, the positive pressure generation unit 120, such as a BiPAP or NIV ventilator, may be configured to increase the generated airflow when the subject is in an inhalation phase of the breath cycle, so as to increase an expiratory volume of the subject.

By performing the therapy at a moderately low intensity, but for a substantial portion of a sleep period (e.g. during high arousal threshold periods), the loosened mucus may be moved more naturally to the mouth by the normal mucocilliary escalator process and swallowed by the patient. This may reduce a burden perceived by patients in the morning and may even reduce or remove the need for additional mucus clearance procedures.

Also, the sleep stage information may be retained and analysed to track progression of a disease and/or to optimize treatment in subsequent nights. This may be especially useful for sleep apnea related treatments.

In a further refinement of this embodiment, to minimize the build-up of loosened mucus in the user's airway, the oscillatory pressure therapy (i.e. OPEP/IPV therapy) may be delivered in a duty cycle, for example ranging from 30%-60%, during a high arousal threshold period. Alternatively, this may be accomplished by prescribing fixed therapy time intervals during high arousal threshold periods. For example, 5, 10 or 15 minutes of oscillatory pressure therapy followed by a pause in the therapy to allow the mucus to be swallowed. In addition, it may preferable to perform oscillatory pressure therapy during high arousal threshold periods closer to the time that the user wakes up so that the loosened mucus does not build up to a level cannot be swallowed by the user naturally. For instance, oscillatory pressure therapy may be ideally performed more frequently in the final one or two sleep stages (e.g. 1-2 hours prior to wake up of the subject). This approach may exhibit higher efficiency, because mucus typically builds up gradually during sleep (thus meaning less mucus loosening is needed during the beginning part of the sleep period).

In the embodiment of FIG. 1, the oscillatory pressure generation unit (16) is configured to be able to modulate the positive pressure airflow at a range of different frequencies. In this way, the oscillation frequency may be varied according to the subject, thus enabling optimisation of the oscillation frequency per subject. For example, the oscillation frequency may be determined based on airway resonance/vibration in the lower chest and upper abdominal region (detected using an accelerometer integrated in a mask worn by the user). In particular, to determine an optimal oscillation frequency, at the start of a therapy session, the system cycles through different frequencies between 11-17 Hz during exhalation to identify the frequency at which the largest amplitude resonance vibration occurs. Once determined, the oscillatory pressure generation unit (16) modulates the airway at the determined frequency, thereby maximizing the efficacy of oscillatory pressure therapy. In a further example, the oscillation frequency may be adjusted based on a measure of the efficacy of the treatment as discussed further below. The oscillation frequency may be adjusted automatically, by way of a controller, or manually by the user.

Such an approach may be especially relevant for therapy during sleep, as relaxation of muscles may yield a different resonance frequency than during an awake period.

It will be appreciated that the proposed concept of superimposing an oscillatory frequency onto a positive pressure airflow may be implemented in different ways. For instance, an oscillatory pressure generation unit may be integrated into a device that is adapted to generate a positive pressure airflow. Such integration may also be done in many ways. Purely by way of example, a blower or turbine of a positive pressure generation unit may be configured to generate pressure fluctuations. This approach may require the blower/turbine to be able to ramp up and down in sub-second intervals in order to generate the required frequencies (e.g. between 11 and 17 Hz). In general, turbines that are capable of this can already be found in NIV ventilators.

Conversely, an oscillatory pressure generation unit may be configured to modulate the positive pressure airflow provided by a conventional positive airway pressure generation unit (such as a conventional CPAP device).

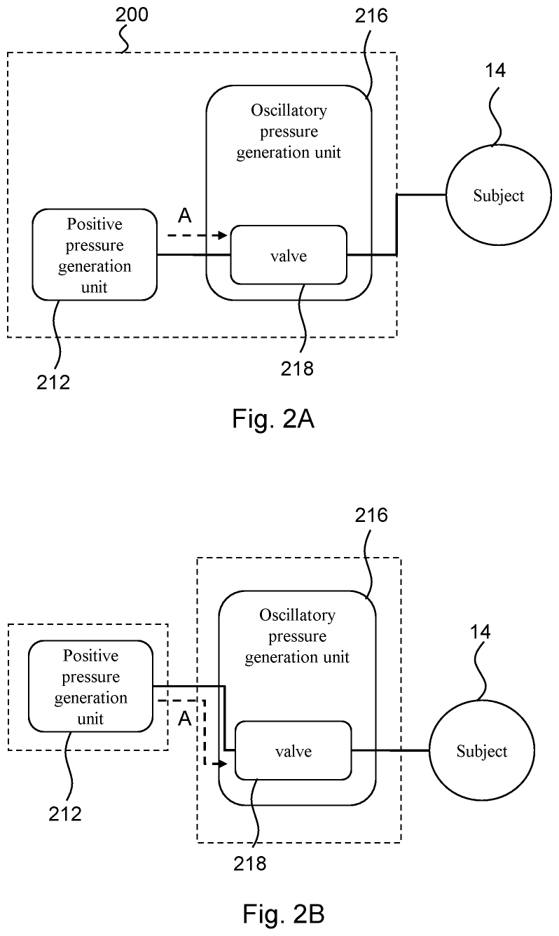

Referring now to FIGS. 2A and 2B, two such exemplary approaches to superimposing an oscillatory frequency onto a positive pressure airflow will now be described.

FIG. 2A is a simplified block diagram of a positive airway pressure therapy system 200 according to an embodiment, wherein an oscillatory pressure generation unit 216 is integrated into a single device along with a positive pressure generation unit 212.

The positive pressure generation unit 212 is adapted to generate a positive pressure airflow ‘A’ for provision to a subject 14 via the oscillatory pressure generation unit 216.

In this embodiment, the oscillatory pressure generation unit 216 comprises an actuator valve 218. The actuator valve 218 is adapted to actuate at the modulation frequency thereby imparting the frequency component to the airflow ‘A’ provided from the positive pressure generation unit 212. The oscillatory pressure generation unit 216 further comprises a pressure sensor (not shown) in communication with the airflow ‘A’. The pressure sensor is adapted to obtain a pressure measurement from the airflow, and the oscillatory pressure generation unit 216 is adapted to adjust the modulation of the airflow ‘A’ based on the pressure measurement.

This approach addresses the issue that some lower-end turbines that may be used in positive pressure generation unit may not have a capacity to generate the required oscillations. By using a (e.g. electrical or pneumatic) valve 218 as depicted in the embodiment of FIG. 2A, the airflow from positive pressure generation unit (which may comprise a conventional CPAP or NIV ventilation device) can be modulated at a required frequency to effectuate oscillatory pressure therapy.

The valve 218 may be placed at various positions in the circuit, for example in the positive airway pressure therapy device itself, or at the subject interface. Due to the weight of the valve, it may be preferable to position the valve 218 inside the positive airway pressure therapy device.

To aid control of the valve 218 (e.g. so that oscillations of a preferred amplitude are created), a feedback loop employing a pressure sensor can be employed. It may therefore be preferable to control the valve 218 with the same processor of the positive airway pressure therapy which also controls the provision of the airflow ‘A’. Further, a pressure sensor may be provided in close proximity to the interface between the subject and the positive airway pressure therapy system, meaning that the amplitude of the pressure oscillations actually received by the subject may be accurately measured. The control of the valve may then be adjusted with greater accuracy based on the measurements received from the pressure sensor in close proximity to the subject. Alternatively, the pressure closest to the interface between the subject and the positive airway pressure therapy system may be estimated by performing a prior calibration of the system between the subject interface and the pressure sensor within the oscillatory pressure generation unit 216. Using the calibration data, the pressure closest to the subject may be estimated from the pressure sensor measurement and a flow sensor within the oscillatory generation unit 216. Thus, in this example an additional flow sensor may be included in the positive airway pressure therapy system in order to calculate the airway resistance. The flow sensor may be provided in the positive pressure generation unit, the oscillatory pressure generation unit or as a separate sensor in communication with the oscillation control processor.

Referring now to FIG. 2B, there is depicted an alternative embodiment, wherein the oscillatory pressure generation unit 216 is provided as a separate device with respect to the positive pressure generation unit 212. That is, the oscillatory pressure generation unit 216 is an add-on device that is placed between the output of the positive pressure generation unit 212 (which may be a conventional CPAP device for example) and the subject 14. This provision of two separate devices is indicated by the two separate dashed boxes indicating the outer boundary/extent of each device.

In this arrangement, the oscillatory pressure generation unit 216 comprises its own processor, pressure sensor, and means for accessing the inhalation/exhalation state of the subject.

From the embodiments illustrated in FIGS. 2A and 2B, it will be understood that a positive pressure generation unit (such as a conventional CPAP, BiPAP or NIV device) may be modified or supplemented to also deliver oscillatory pressure therapy.

By integrating an oscillatory pressure generation unit with a positive pressure generation unit, a single device that delivers concurrent positive pressure airflow and oscillatory pressure therapy can be provided, thus avoiding a need for a subject to use (e.g. support, operate and hold) separate devices during therapy.

By employing a separate oscillatory pressure generation unit, a conventional/legacy positive pressure generation unit may supplemented/augmented to provide additional functionality (such as oscillatory pressure therapy). Embodiments may therefore extend or improve the functionality of existing positive pressure generation units, such as existing CPAP and NIV devices.

It is noted that the oscillatory pressure therapy mode does not always need be activated. Rather, it may be enabled by the user/subject, who can schedule therapy around normal CPAP/NIV usage (e.g. prior to sleeping and after waking up), or at separate moments during the day.

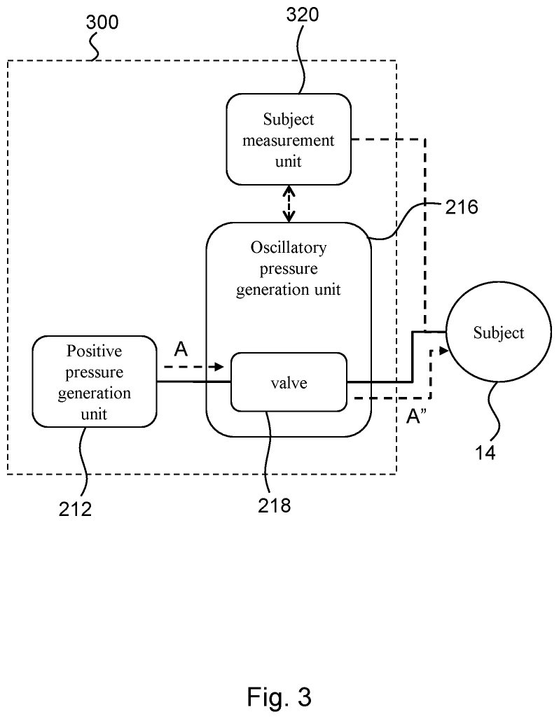

Referring now to FIG. 3, there is depicted a simplified block diagram of a positive airway pressure therapy system 300 according to another embodiment. Specifically, the system 300 of FIG. 3 is a modified version of the embodiment of FIG. 2A, wherein the system 300 further comprises a subject measurement unit 320 adapted to acquire a measure from the subject.

The subject measurement unit 320 is adapted to obtain an efficacy measure of the modulated airflow A″ from the subject 14. Based on the efficacy measure, the oscillatory pressure generation unit 216 is adapted to adjust the modulation of the airflow ‘A’.

Such a feedback concept may benefit from a method to detect the work done, i.e. the amount of mucus swallowed. This could, for example, be performed by detecting the sound of swallowing events, but also by detecting disturbances to the airflow and pressure waveforms of the airflow (since swallowing would coincide with glottis closure and therefore will interrupt the inspiratory and expiratory flow and disturb the device operation). By counting the swallowing events, a measure of therapy effectiveness (in terms of clearance) may be derived. This measure could be used to optimize mucus mobilization over multiple nights (e.g. by testing different frequency/pressure ranges during sleep and recording the outcome).

Further, in this embodiment 320, the subject measurement unit is adapted to obtain a measure of airway resistance from an airway of the subject. Based on the measure of airway resistance, the oscillatory pressure generation unit 216 modulates the airflow ‘A’.

In yet another embodiment, it is proposed to trigger oscillatory pressure therapy during the subject's sleep cycle when a specific threshold of mucus build-up has been reached or exceeded. The build-up of mucus is detected based on changes in the airway resistance, which can be non-invasively measured by a ventilator. The airway resistance is defined as the differential pressure, measured between the output of the positive pressure generation unit and the interface between the system and the subject (such as the ventilation mask), divided by the airflow through the system. The differential pressure and airflow may be measured over time. Once mucus build-up is detected, the oscillatory pressure generation unit is activated to loosen the mucus when the user is in the next high arousal threshold sleep stage. In this example an additional flow sensor may be included in the positive airway pressure therapy system in order to calculate the airway resistance. The flow sensor may be provided in the positive pressure generation unit, the oscillatory pressure generation unit or as a separate sensor in communication with the oscillation control processor.

That is, according to proposals, the subject measurement unit 320 may be adapted to obtain a respiratory mechanics measure from an airway of the subject, and the oscillatory pressure generation unit 216 may then be adapted to adjust modulation of the airflow ‘A’ based on the respiratory mechanics measure.

While the main intention of oscillatory pressure therapy is to help in mucus clearance, the frequency content of the oscillatory pressure therapy may also be utilized to estimate respiratory mechanics (e.g. resistance and compliance). This can be done by either a single oscillation frequency, or by sweeping through a range of frequencies. Respiratory mechanics can then be derived.

For example, a sweep of multiple frequencies may assist in the selection of an optimal oscillation frequency range or optimal single oscillation frequency (e.g. by plotting the impedance spectra as a function of frequency). The optimal frequency range or single frequency which minimizes impedance or results in resonance may be checked automatically with time and applied.

Also, with the ability to measure resistance and compliance over time, treatment assessment as well as continuous monitoring of patient status may be achieved.

Yet further, an accelerometer or other body posture sensing device may be employed to detect subject posture(s) in which mucus mobilisation is more effective. The subject may then be advised/trained to adopt an optimal posture.

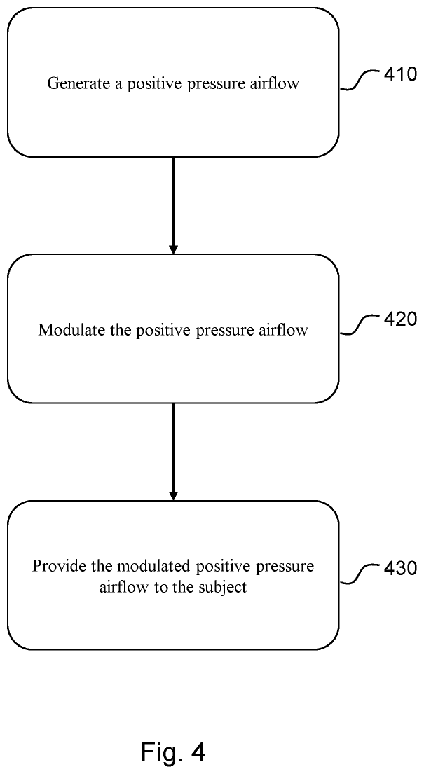

Referring now to FIG. 4, there is depicted a flow diagram of a positive airway pressure therapy method 400 according to an embodiment.

The method begins with step 410 of generating a positive pressure airflow for provision to a subject (14). Next, in step 420, the positive pressure airflow is modulated at a modulation frequency, thereby imparting a frequency component to the positive pressure air flow to be provided to the subject. Here, the airflow is modulated (at least) during an exhalation phase of a breathing cycle of the subject. Put another way, pressure oscillations are imparted onto the positive pressure air flow to be provided to the subject.

Finally, in step 430, the modulated positive pressure airflow is provided to the subject.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A positive airway pressure therapy system comprising:
a positive pressure generation unit adapted to generate a positive pressure airflow for provision to a subject; and
an oscillatory pressure generation unit adapted to modulate the positive pressure airflow at a modulation frequency thereby imparting a frequency component to the positive pressure air flow to be provided to the subject, wherein the oscillatory pressure generation unit is adapted to modulate the airflow during an exhalation phase of a breathing cycle of the subject.

2. The positive airway pressure therapy system claimed in claim 1, wherein the oscillatory pressure generation unit is further adapted to modulate the airflow during a predetermined sleep stage of the subject.

3. The positive airway pressure therapy system claimed in claim 2, wherein the oscillatory pressure generation unit is further adapted to generate a sleep trend over a plurality of sleep sessions, and wherein the oscillatory pressure generation unit is further adapted to adjust modulation of the airflow based on the sleep trend.

4. The positive airway pressure therapy system as claimed claim 1, wherein the modulation frequency comprises a range of frequencies.

5. The positive airway pressure therapy system as claimed in claim 1, wherein the oscillatory pressure generation unit comprises an actuator valve, wherein the actuator valve is adapted to actuate at the modulation frequency thereby imparting the frequency component to the airflow.

6. The positive airway pressure therapy system as claimed in claim 5, wherein the oscillatory pressure generation unit further comprises a pressure sensor in communication with the airflow, wherein the pressure sensor is adapted to obtain a pressure measurement from the airflow, and wherein the oscillatory pressure generation unit is adapted to adjust the modulation of the airflow based on the pressure measurement.

7. The positive airway pressure therapy system as claimed in claim 1, wherein the oscillatory pressure generation unit is adapted to control the positive pressure generation unit to modulate the airflow.

8. The positive airway pressure therapy system as claimed claim 1, wherein the oscillatory pressure generation unit and the positive pressure generation unit are integrated into a ventilation device.

9. The positive airway pressure therapy system as claimed claim 1, wherein the positive pressure generation unit is further adapted to increase the generated airflow when the subject is in an inhalation phase of the breath cycle.

10. The positive airway pressure therapy system as claimed in claim 1, further comprising a subject measurement unit adapted to acquire a measure from the subject.

11. The positive airway pressure therapy system as claimed in claim 10, wherein the subject measurement unit is adapted to obtain an efficacy measure of the modulated airflow from the subject, and wherein the oscillatory pressure generation unit is adapted to adjust the modulation of the airflow based on the efficacy measure.

12. The positive airway pressure therapy system as claimed in claim 10, wherein the subject measurement unit is adapted to obtain a measure of airway resistance over time from an airway of the subject, and wherein the oscillatory pressure generation unit is adapted to modulate the airflow based on the measure of airway resistance.

13. The positive airway pressure therapy system as claimed in claim 10, wherein the subject measurement unit is adapted to obtain a respiratory mechanics measure from an airway of the subject, and wherein the oscillatory pressure generation unit is adapted to adjust the modulation of the airflow based on the respiratory mechanics measure.

14. The positive airway pressure therapy system as claimed in claim 10, wherein the subject measurement unit is adapted to obtain a measurement of a posture of the subject, and wherein the oscillatory pressure generation unit is adapted to adjust the modulation of the airflow based on the posture of the subject.

15. The positive airway pressure therapy system as claimed in claim 1, wherein the positive airway pressure therapy system comprises one or more of:
a CPAP device;
a BiPAP device; and
an NIV device.
